(19) United States
(12) Patent Application Publication
(10) Pub. No.: US 2012/0231016 A1
(43) Pub. Date: Sep. 13, 2012

(54) Pharmaceutical Composition for Inhibiting Histone H4 and Manufacturing Method Thereof

(75) Inventors: Min-Jen Tseng, Minxiong Township (TW); Ching-Wu Hsia, Taipei City (TW); Ko-Ta Cheng, New Taipei City (TW); Chun-Mao Lin, New Taipei City (TW)

(73) Assignees: NATIONAL CHUNG CHENG UNIVERSITY, Minhsiung Township (TW); TAIPEI MEDICAL UNIVERSITY, Taipei City (TW)

(21) Appl. No.: 13/406,209

(22) Filed: Feb. 27, 2012

(30) Foreign Application Priority Data
Mar. 11, 2011 (TW) 100108440

(51) Int. Cl. A61K 39/395; A61P 17/14

(52) U.S. Cl. 424/172.1

(57) ABSTRACT

The present invention discloses a pharmaceutical composition for inhibiting histone H4 and a manufacturing method thereof. The pharmaceutical composition comprises an anti-histone H4 antibody and a pharmaceutically acceptable excipient. The hair loss resulted from medications, including cancer drugs, can be reduced by using the pharmaceutical composition of the present invention, thereby resolving mental depression and promoting prognosis effects for patients.

### FIELD OF THE INVENTION

This application claims the benefit of Taiwan Patent Application No. 100108440, filed Mar. 11, 2011, the contents of which are hereby incorporated by reference in their entirety for all purposes.

The present invention is directed to a pharmaceutical composition and a manufacturing method thereof, particularly to a pharmaceutical composition for inhibiting histone H4 and a manufacturing method thereof.

### BACKGROUND OF THE INVENTION

Hair is produced from hair follicles through generation of protein, that is to say no hair grows without hair follicles. The growth cycle of hair proceeds through three phases: anagen, catagen and telogen, in this order. The anagen phase physiologically functions as making hair shaft, and is divided into six subphases according to types of hair follicles. The span of the sixth subphase determines the length of hair shaft. When the anagen phase begins, initial change of hair follicles is occurring between dermal papilla of a hair follicle and hair germs from another hair follicle of the same phase. Epithelial cells of the hair germs grow downwardly along dermal tail produced in the previous telogen phase, and reaches dermis to produce epidermal fingers. Then, the central cells of the hair germ grow to produce the hair shaft and the inner root sheath (IRS), and the stratum basal cells of the hair follicle divide along Auber's critical level of dermal papilla cells.

When the hair follicles are at late catagen phase, the cyclic structure deteriorates and disappears, and only the permanent part and dermal papilla cells exist. In the catagen phase, the changes in hair follicles include that hair shaft making stops, no pigment is produced from melanocytes, a great quantity of basal cells differentiate into apoptosis, dermal papilla cells shrink, extracellular matrix environment of dermal papilla cells disappear, and finally hair follicles shrink in vertical direction.

The mechanism that how hair follicles at the anagen phase are affected by signal transmission molecules to enter into the catagen phase is yet to be known. However, environmental factors like chemicals, serious psychological stress, trauma, etc. can accelerate this process. In addition, the major physiological change of hair follicles during the catagen phase is cell apoptosis.

In the telogen phase, both epithelial cells of the permanent part and dermal papilla cells rest, peripheral vascular tissue disappears, and hair shaft is covered by vesicula formed of two layers of epithelial cells to produce club hair. Cells between bottom of the club hair and the vesicular epithelial cells are so called hair germ. As currently limited literatures about hair follicles at the telogen phase have been reported, it is only known that elimination of desmoglein-3 and cathepsin L causes abnormal formation of hair shaft during the telogen phase. When hair follicles at the telogen phase enter into next anagen phase, the hair germ cells become larger and grow downwardly to approach dermal papilla cells, and new hair shaft will emerge at the same position once the original hair falls.

Drug-induced alopecia/effluvium is one of the serious side effects commonly seen clinically. Medicines causing alopecia include anticancer (chemotherapeutic) drugs, antibiotics, alkaloids, etc. Currently the side effect known as chemotherapy-induced alopecia (CIA) is one of the frequent medicine clinical issues to be solved. Some chemotherapeutic drugs are liable to kill both cancer cells and fast-dividing normal human cells, so that cancer patients suffer from hair loss and in turn are into serious mental depression, inadaptable to usual family and social lives, which at some level influences their prognosis effects, even the willingness to complete the chemotherapy. Therefore, it is required to find out the causes of the drug-induced alopecia/effluvium and to improve clinically.

### SUMMARY OF THE INVENTION

In regard to the issues in prior art as discussed above, the objects of the present invention is to provide a pharmaceutical composition for inhibiting histone H4 and a manufacturing method thereof to alleviate the side effect of drug-induced alopecia/effluvium.

An object of the present invention is to provide a pharmaceutical composition for inhibiting histone H4 comprising an anti-histone H4 antibody and a pharmaceutically acceptable excipient.

The object of the present invention is further to provide a manufacturing method of a pharmaceutical composition for inhibiting histone H4, which comprises a step of mixing an anti-histone H4 antibody and a pharmaceutically acceptable excipient to produce the pharmaceutical composition for inhibiting histone H4.

The composition according to the present invention is useful in drug-induced alopecia/effluvium. The drugs may be anticancer drugs including colchicine, adriamycin, carboplatin, paciltaxel or taxol. The pharmaceutically acceptable excipient is selected from a group consisting of flavoring agent, preservative, antioxidant, chelating agent, isotonic agent, tablet adjuvant, colorant, binding agent, and pharmaceutically acceptable carrier. Additionally, the composition according to the present invention can be made into the form of powder, tablet, emulsion, gel or spray, in order to adapt to various types of users and administration ways.

From the above, the pharmaceutical composition for inhibiting histone H4 and the manufacturing method thereof according to the present invention comprise one or more advantages as following.

(1) When dermal papilla cells are stimulated by medicines, histone H4 is autocrined and in turn inhibits the dermal papilla cells and reduces the alkaline phosphatase activity of the dermal papilla cells. Thus, the pharmaceutical composition according to the present invention blocks all influence caused by histone H4 on dermal papilla cells and reduces hair loss due to inclusion of anti-histone H4 antibody.

(2) As the pharmaceutical composition according to the present invention is useful in alleviating the side effects of drug-induced alopecia/effluvium, mental depression caused by hair loss and inadaptability to usual family and social life are moderated, the prognosis effects can be promoted.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 are staining photographs showing dermal papilla cells obtained from vibrissa hair of Sprague-Dawley (SD) rats in vitro;

FIG. 2A shows the effect of 12.5% colchicine conditional medium (CCM) on proliferation of dermal papilla cells;

FIG. 2B shows the effect of 33% CCM on proliferation of dermal papilla cells;

FIG. 2C shows the effect of 50% CCM on proliferation of dermal papilla cells;

FIG. 2D shows the effects of 2-μM-6h-CCM at various concentrations cultured for 72 and 120 hours on proliferation of dermal papilla cells;

FIG. 3 shows the result of cell apoptosis and cell cycle from cells processed with various 5% medium for 72 hours and then stained with propidium iodide (PI) fluorescent agent, analyzed by Flow Cytometry;

FIG. 4A shows the result of effect on the alkaline phosphatase activity of 2-μM-6h-CCM;

FIG. 4B shows the result of effect on the mRNA expression of alkaline phosphatase of 2-μM-6h-CCM;

FIG. 5A shows the analytical result of parting liquid from molecular sieve centrifuge tubes on proliferation of dermal papilla cells;

FIG. 5B is the RPC₈—HPLC chromatogram of 5-10 kDa 2-μM-6h-CCM (fraction 2);

FIG. 5C is the SEC-HPLC chromatogram of Peak 2;

FIG. 5D shows the analytical results of 5% Peak 2-1 and Peak 2-2 purified substances on proliferation of dermal papilla cells;

FIG. 6A shows the analytical results of 2-μM-6h-CCM and Peak 2-2 purified substance on thermal stability;

FIG. 6B shows the analytical results of 2-μM-6h-CCM and Peak 2-2 purified substance on trypsin sensitivity;

FIG. 7 is the 15% sodium dodecylsulfate-polyacrylamide gel electrophoresis (SDS-PAGE) diagram of Peak 2-2 purified substance;

FIGS. 8A-8E are the mass spectrum of fragments from Peak 2-2 purified substance after trypsin cleaving;

FIG. 9 shows the analytical result of Peak 2-2 purified substance by Western blot method;

FIG. 10A shows the analytical result of recombinant histone H4 on proliferation of dermal papilla cells; and

FIG. 10B shows the analytical results of 2-μM-6h-CCM and Peak 2-2 purified substance on proliferation of dermal papilla cells.

### DETAILED DESCRIPTION OF THE INVENTION

The present invention is described in detail with reference to the following preferred embodiments in conjunction with the accompanying drawings. It is appreciated that experimental data shown in the following embodiments are used to explain technical features of the present invention without limiting the practical aspects in any way.

The pharmaceutical composition for inhibiting histone H4 according to the present invention comprises an anti-histone H4 antibody and a pharmaceutically acceptable excipient. The present composition is useful in treating hair loss resulted from medications, including anticancer drugs. In addition, the manufacturing method of the pharmaceutical composition for inhibiting histone H4 according to the present invention comprises a step of mixing an anti-histone H4 antibody and a pharmaceutically acceptable excipient to produce a pharmaceutical composition for inhibiting histone H4.

The pharmaceutically acceptable excipient mentioned above is selected from a group consisting of flavoring agent, preservative, antioxidant, chelating agent, isotonic agent, tablet adjuvant, colorant, binding agent, and pharmaceutically acceptable carrier. Also, the composition according to the present invention can be made into the form of powder, tablet, emulsion, gel or spray, in order to meet various requirements from users.

Drug-induced alopecia/effluvium is one of the serious side effects commonly seen clinically. Clinical responses of hair follicles to anticancer drugs are diverse. For example, high usage of adriamycin for 3-4 weeks leads to sparse eyebrows and eyelashes; combination use of carboplatin and cyclophosphamide leads to hair loss; and use of either paciltaxel or taxol for 2-3 weeks causes 100% of cancer patients to lose hair. Further, use of colchicine in any dosage not only changes hair condition but also leads to alopecia.

Therefore, the following examples are provided to show the effects of anticancer drugs on dermal papilla cells, and by co-culture of colchicine with dermal papilla cells to determine whether the drugs cause dermal papilla cells to autocrine histone H4, and further to obtain the effects of histone H4 on proliferation and alkaline phosphatase activity of peripheral cells. It is noted that, although the anticancer drug used in examples is colchicine, it is not limited thereto and others like adriamycin, carboplatin, cyclophosphamide, paciltaxel, etc. are equivalently useful. In addition, the control groups shown in the accompanying drawings illustrate normal dermal papilla cells. Data statistics for the following tests is paired t-test. If data is statistically significant, the symbol “*” is used to indicate p-value. The symbol “*” indicates p<0.05 and the symbol “**” indicates p<0.01.

(1) Preparation of Colchicine Solution

Colchicine is dissolved in dimethyl sulfoxide (DMSO) to prepare 10 mM colchicine solution. Then, the solution is distributed under aseptic condition and stored in refrigerator at −20° C.

(2) Culture of Dermal Papilla Cells In Vitro

Dermal papilla cells are collected from vibrissa hair of 8-week-old male SD rats under dissecting microscope with microsurgery. Zeroth generation of dermal papilla cells (DPCs) are cultured in a 35 mm dish with the RPMI 1640 medium, and then 10% heat-treated fetal calf serum, 2 mM L-glutamine, 50 IU/ml penicillin and 50 mg/ml streptomycin are added. When the cells grew to fill 80% of the dish, the cells are re-cultured in a new 100 mm culture dish and are marked as first generation of dermal papilla cells. The dermal papilla cells used in examples are the sixth or earlier generations. The dermal papilla cells are cultured in the cell incubator at 37° C. with the addition of 5% CO₂.

(3) Preparation of Colchicine Conditional Medium without Serum

5×10^{6/5} ml of the dermal papilla cells are incubated with colchicine solutions at various concentrations, and then are washed three times with PBS buffer for 5 minutes to remove residual colchicine. Finally, the dermal papilla cells are cultured in 5 ml of RPMI 1640 medium with 50 IU/ml penicillin and 50 mg/ml streptomycin for 12 hours. The supernatant is collected to use as “colchicine conditional medium”, and is distributed and stored in refrigerator at −80° C.

(4) Alkaline Phosphatase (ALP) Activity of Dermal Papilla Cells

Dermal papilla cells having strong activity exhibit high alkaline phosphatase activity. When the dermal papilla cells rest, the alkaline phosphatase activity gradually decreases. Thus, the expression of the alkaline phosphatase can be used as primary index to obtain the cell activity. 1 ml of the dermal papilla cells (with cell density of 1000/ml) are cultured in each hole of a 24-hole culture plate to proceed alkaline phosphatase staining. The dermal papilla cells are firstly fixed with buffered acetone consisting of 1.92 mM sodium citrate, 0.08 mM citric acid and 60% of acetone at room temperature for 45 seconds. Then, fixed dermal papilla cells are washed by large quantity of deionized water. Further, the dermal papilla cells are incubated in culture solution (22 mg of fast blue BBN salt/5 ml of naphthol phosphate buffer) at room temperature for 30 minutes in the dark, and are washed by large quantity of deionized water. The dermal papilla cells are stained with 0.5% ethanol solution of safranin O for 45 seconds, and then are washed with large quantity of deionized water. Finally, the dermal papilla cells are dried in the air. Blue dermal papilla cells are positive for the alkaline phosphatase activity. The stained dermal papilla cells are photographed, and number of dermal papilla cells with alkaline phosphatase activity is counted.

(5) Activity Analysis on Colchicine Conditional Medium:

2-μM-6h-CCM inhibits proliferation of dermal papilla cells, but cell apoptosis is not intensified and cell cycle is not affected.

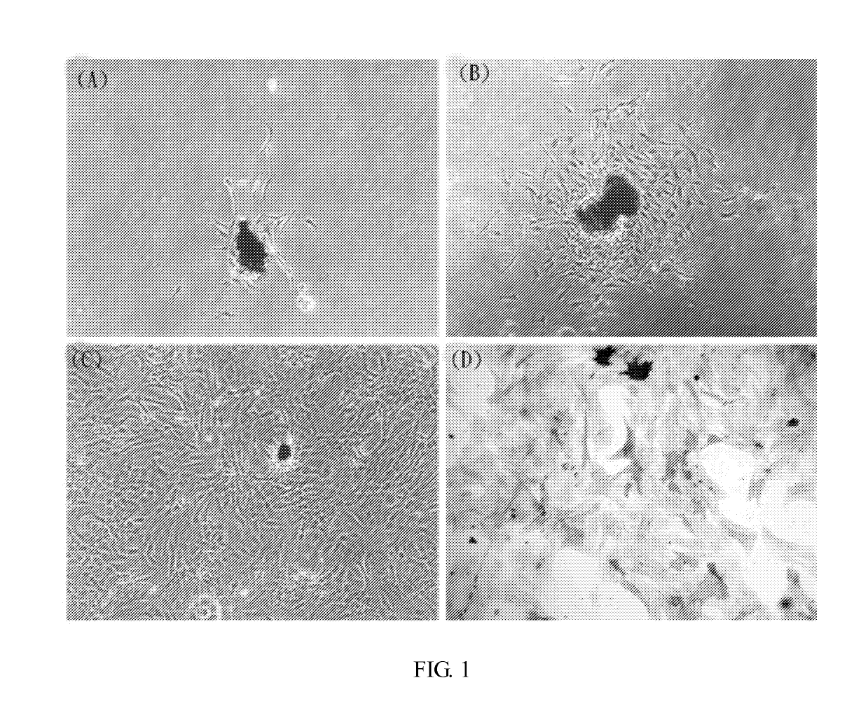

FIG. 1 are the staining photographs showing the dermal papilla cells obtained from vibrissa hair of male SD rats with microscopy surgery in vitro. FIG. 1A is taken from zeroth generation in vitro on Day 5 and is magnified to 200×; FIG. 1B is taken from zeroth generation in vitro on Day 10 and is magnified to 200×; FIG. 1C is taken from zeroth generation in vitro on Day 28 and is magnified to 200×; and FIG. 1D shows the result of the alkaline phosphatase activity staining magnified to 400×, in which blue areas represent the dermal papilla cells with the alkaline phosphatase activity. It is seen from FIG. 1 that the dermal papilla cells with the alkaline phosphatase activity are successfully extracted from the vibrissa hair of male SD rats in vitro.

To further investigate the conditions that colchicine affects the dermal papilla cells to secrete various factors, the dermal papilla cells are incubated with 0, 0.1, 1 and 2 μM colchicine solutions for 1, 2, 3 and 6 hours to prepare colchicine conditional medium (CCM). After the dermal papilla cells are treated with various proportion of CCM for 72 or 120 hours, numbers of proliferated cells are counted. For ease of reading, the above culture conditions are described in abbreviated forms; for example, “CCM prepared by incubating the dermal papilla cells with 2 μM colchicine solutions for 6 hours” is described as “2-μM-6h-CCM”.

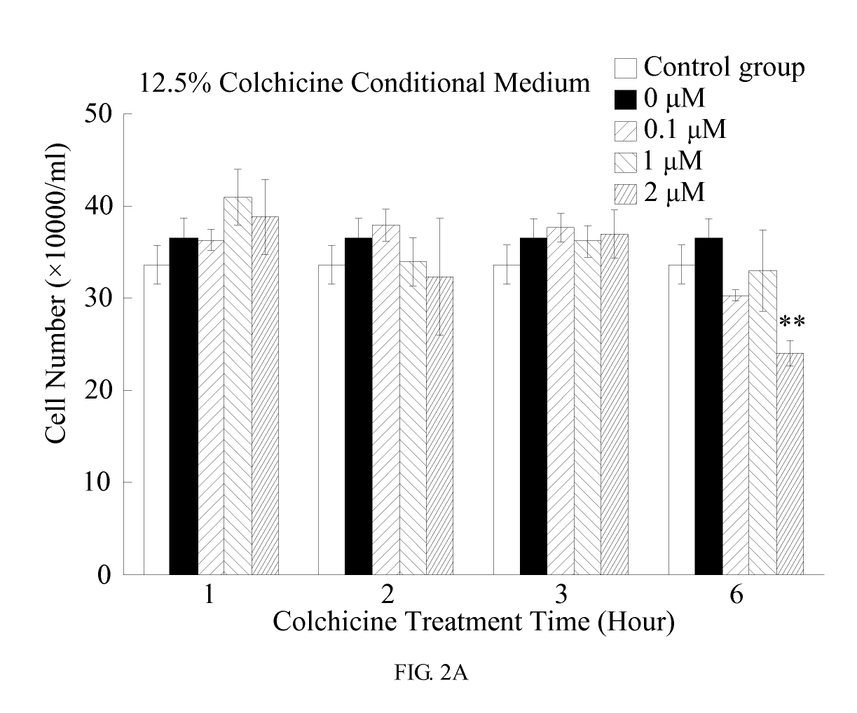

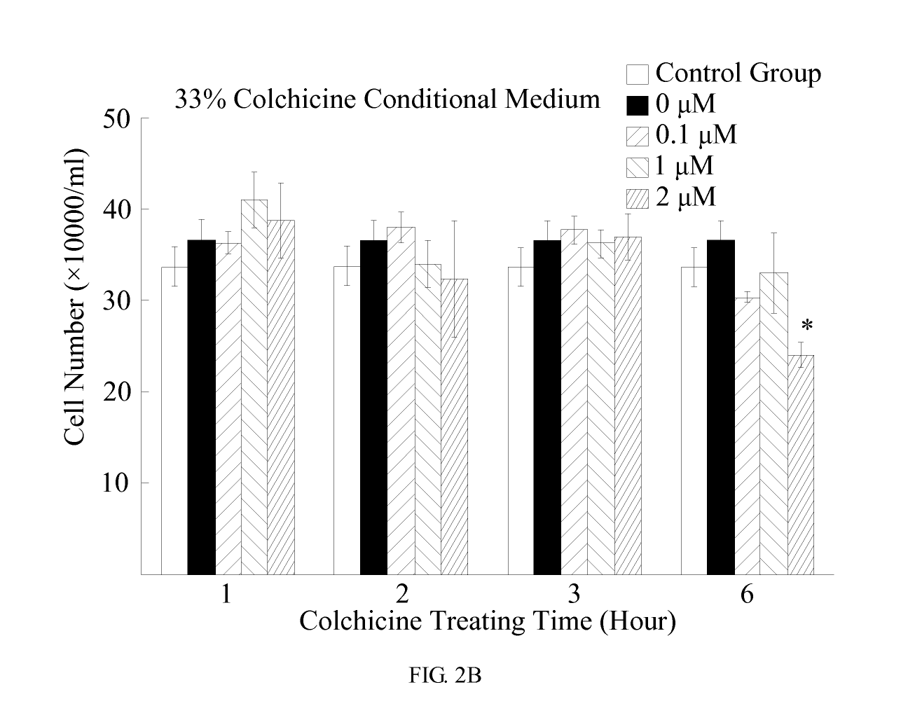

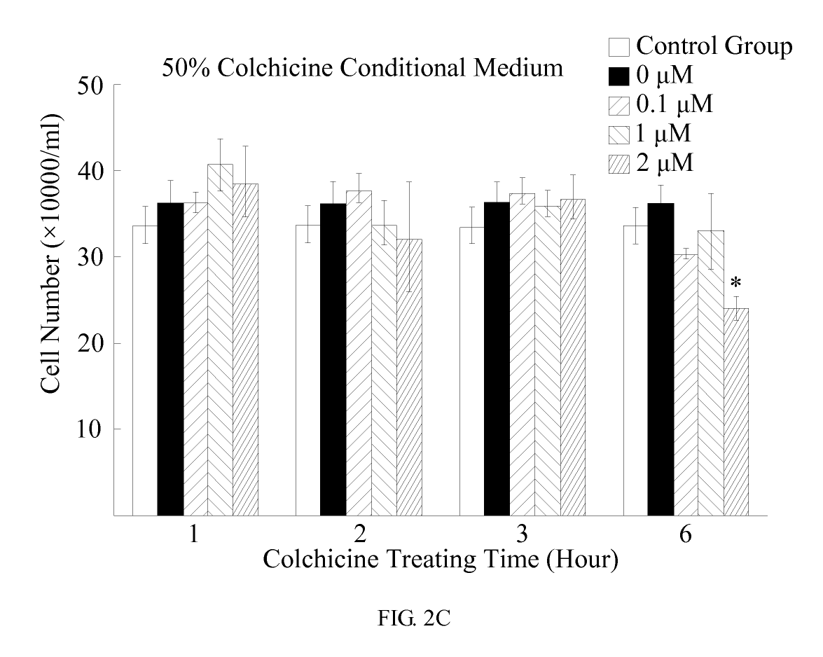

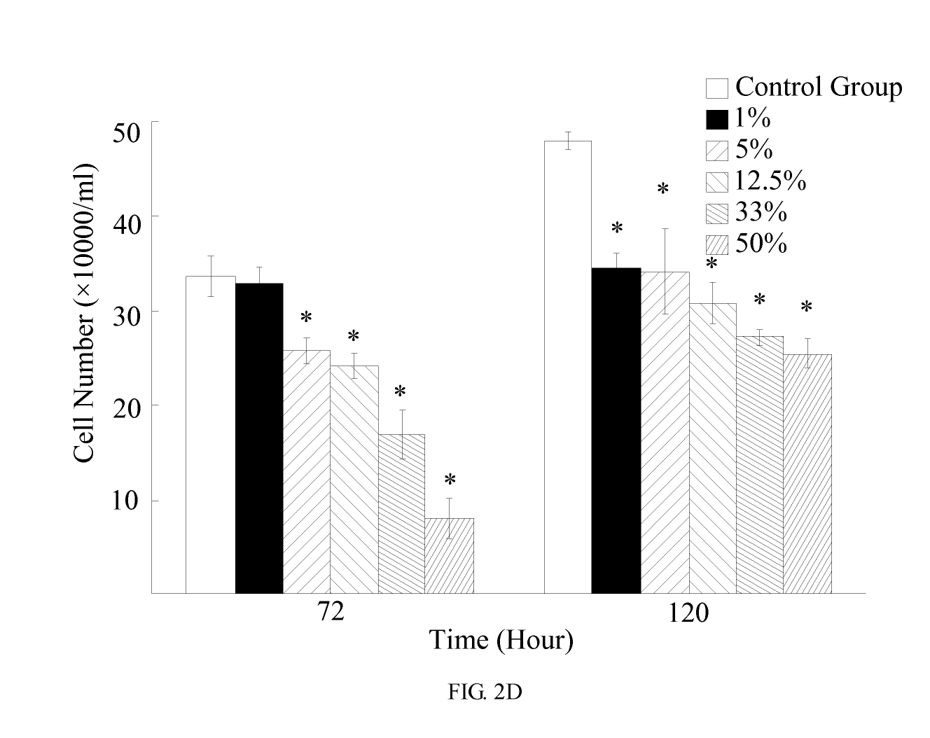

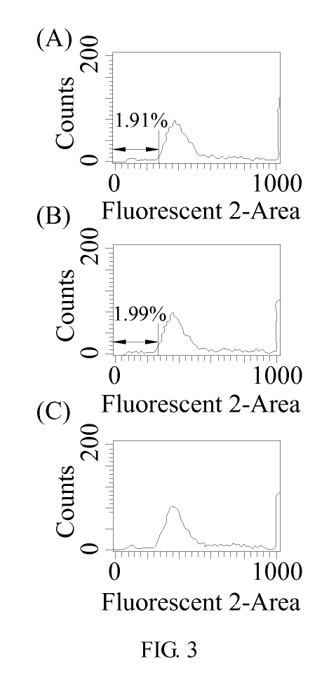

It is found that the proliferation of dermal papilla cells is inhibited only by 2-μM-6h-CCM, as shown in FIGS. 2A, 2B and 2C. The inhibition level of proliferation is proportional to the dosage of 2-μM-6h-CCM, and the lowest inhibition concentrations are 5% (72 hours) and 1% (120 hours), as shown in FIG. 2D. The dermal papilla cells incubated with 5% 2-μM-6h-CCM for 72 hours are stained with propidium iodide (PI) fluorescent agent and analyzed by Flow Cytometry. The result is shown in FIG. 3, wherein the mediums used in FIGS. 3A, 3B and 3C are RPMI 1640, 0-μM-CCM and 2-μM-6h-CCM, respectively. It can be seen that though 5% 2-μM-6h-CCM inhibited the proliferation, the cell apoptosis (sub-G1) is not intensified and cell cycle (G2/M) is not changed.

(6) Separation and Purification of Inhibition Factor:

2-μM-6h-CCM inhibits proliferation of dermal papilla cells and reduces alkaline phosphatase activity and alkaline phosphatase mRNA expression.

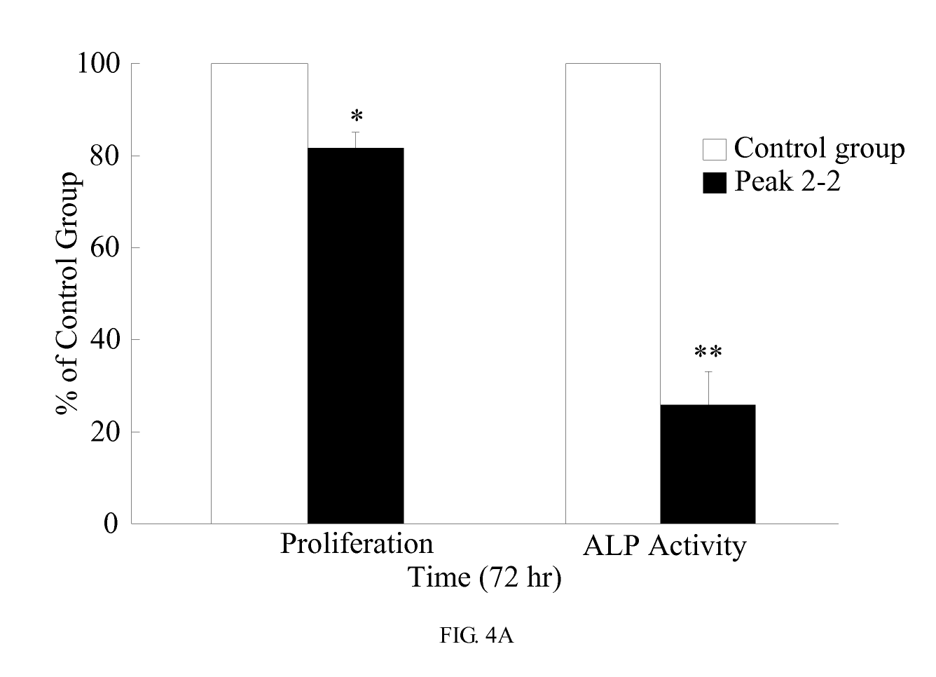

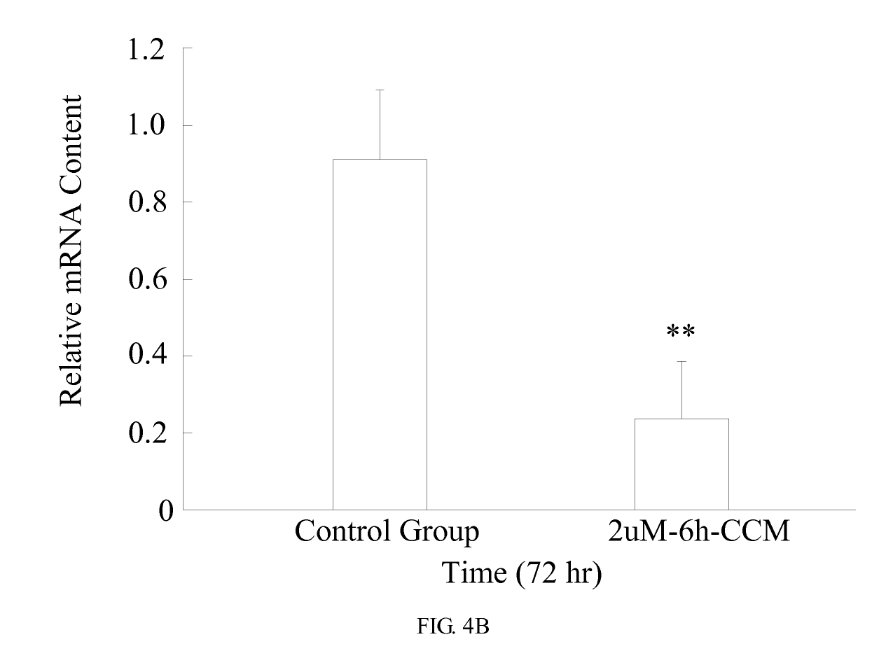

To further investigate how 2-μM-6h-CCM inhibits the proliferation of the dermal papilla cells, firstly the effect of 2-μM-6h-CCM on the alkaline phosphatase activity of the dermal papilla cells is examined by the alkaline phosphatase activity staining. It is found that 5% 2-μM-6h-CCM inhibited 18.5% of proliferation and 74.1% of alkaline phosphatase activity of the dermal papilla cells, as shown in FIG. 4A. The effect of 2-μM-6h-CCM on the alkaline phosphatase activity of the dermal papilla cells is further examined by using an expanded quantitative polymerase test (real-time quantitative polymerase chain reaction, real-time PCR or qPCR), and it is found that 5% 2-μM-6h-CCM is useful in inhibiting 61% of alkaline phosphatase mRNA expression, as shown in FIG. 4B.

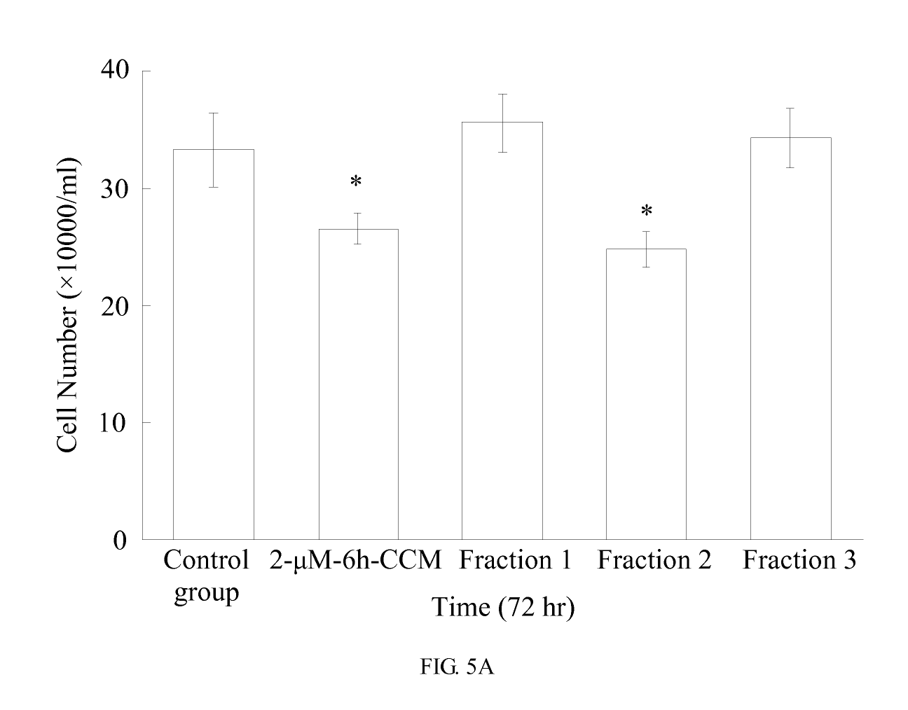

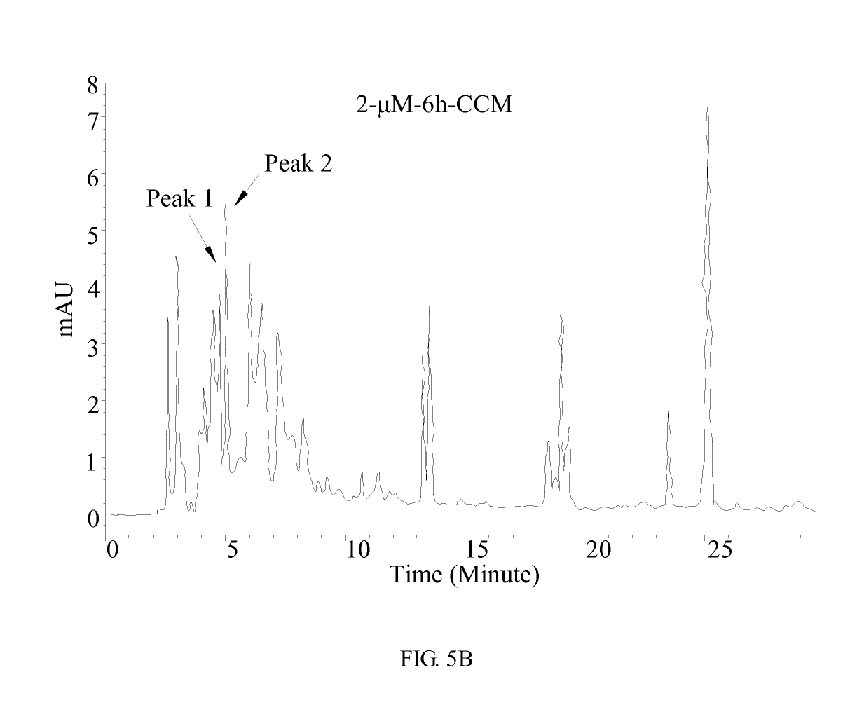

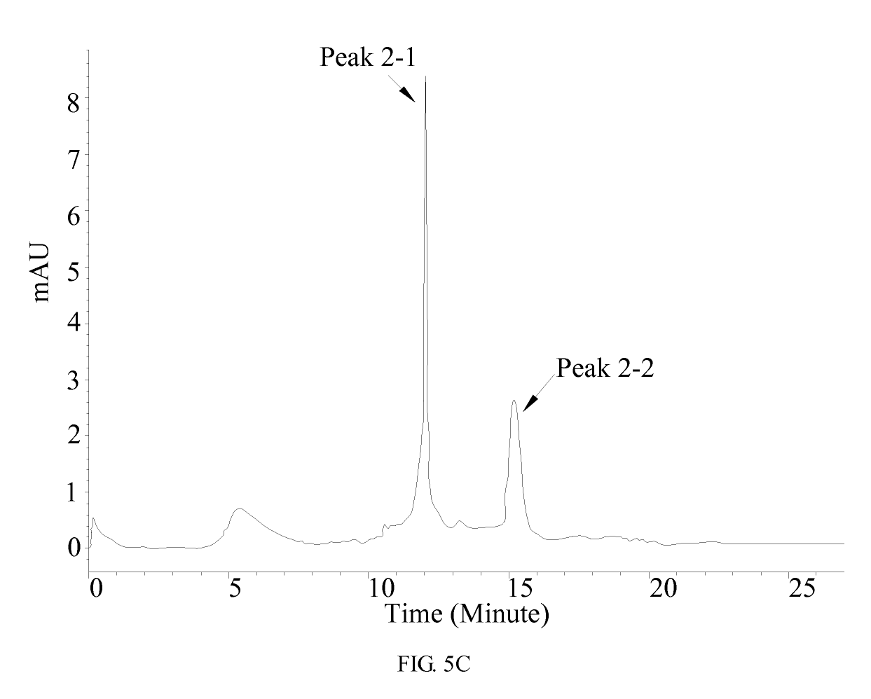

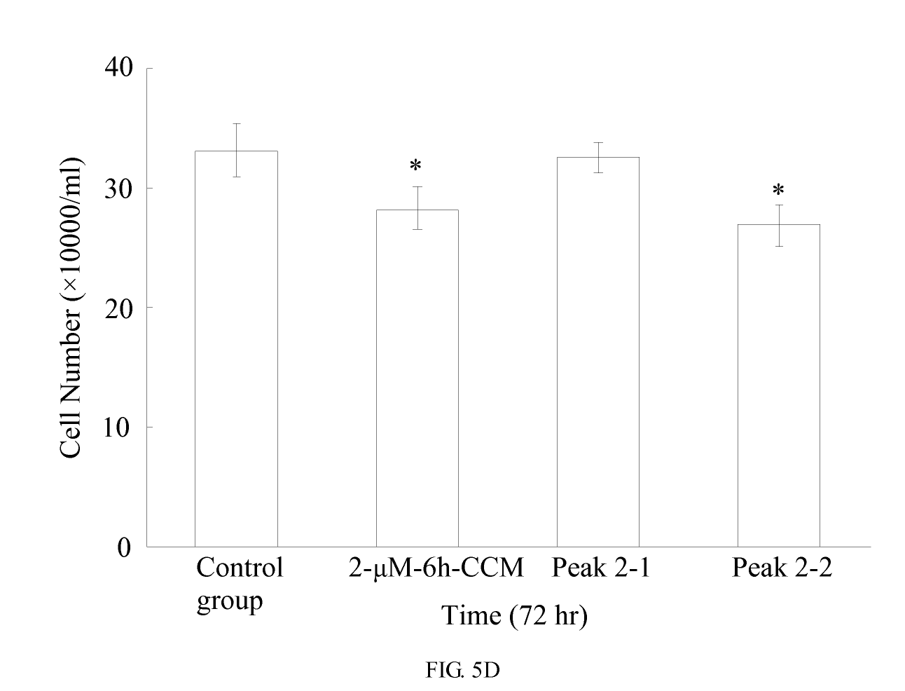

2-μM-6h-CCM is separated by the MWCO (molecular weight cutoff) molecular sieve centrifuge tube, and the molecular weight ranges of various factors secreted from the dermal papilla cells affected on colchicine are determined. It is found that 5% 5-10 kDa 2-μM-6h-CCM (i.e. fraction 2) is capable of inhibiting proliferation (at about 25.5%), as shown in FIG. 5A. In summary, the molecular weight of growth inhibitor in 2-μM-6h-CCM is between 5 to 10 kDa.

Therefore, to find out the structure, firstly the growth inhibitors in 5-10 kDa 2-μM-6h-CCM (fraction 2) are purified by the reverse phase C8 column high performance liquid chromatography (RPC₈ HPLC). Two peaks with particularly high absorbance appear in the RPC₈—HPLC chromatogram at retention time (T_{R})=4.765 min for Peak 1 and 5.013 min for Peak 2, as shown in FIG. 5B.

The result of the growth examination of the dermal papilla cells shows that only Peak 2 purified substance is capable of inhibiting growth of the dermal papilla cells (at about 18.5%). It is further analyzed with size exclusion chromatography-high performance liquid chromatography (SEC-HPLC). The result is shown in FIG. 5C in which Peak 2 is separated into 2 peaks (Peak 2-1: T_{R}=12.205 min; and Peak 2-2: T_{R}=15.169 min). Subsequently, it is found from the growth examination that only Peak 2-2 purified substance is capable of inhibiting growth of the cells (at about 20.6%), as shown in FIG. 5D.

(7) Identification and Verification of Inhibitor:

Peak 2-2 is not thermal stable and is sensitive to trypsin, and is identified as histone H4 by Western blot method.

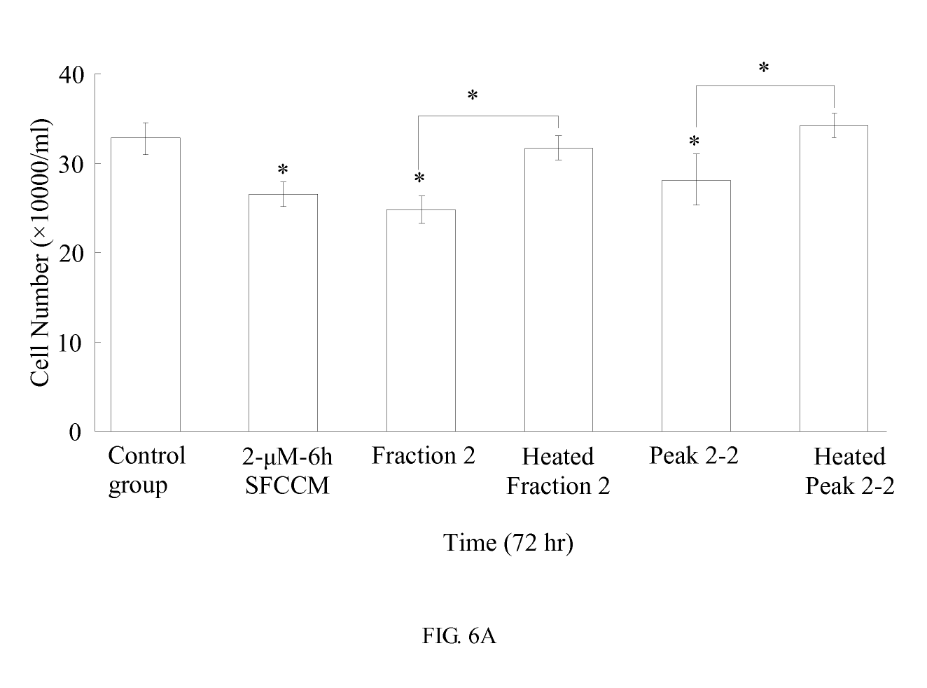

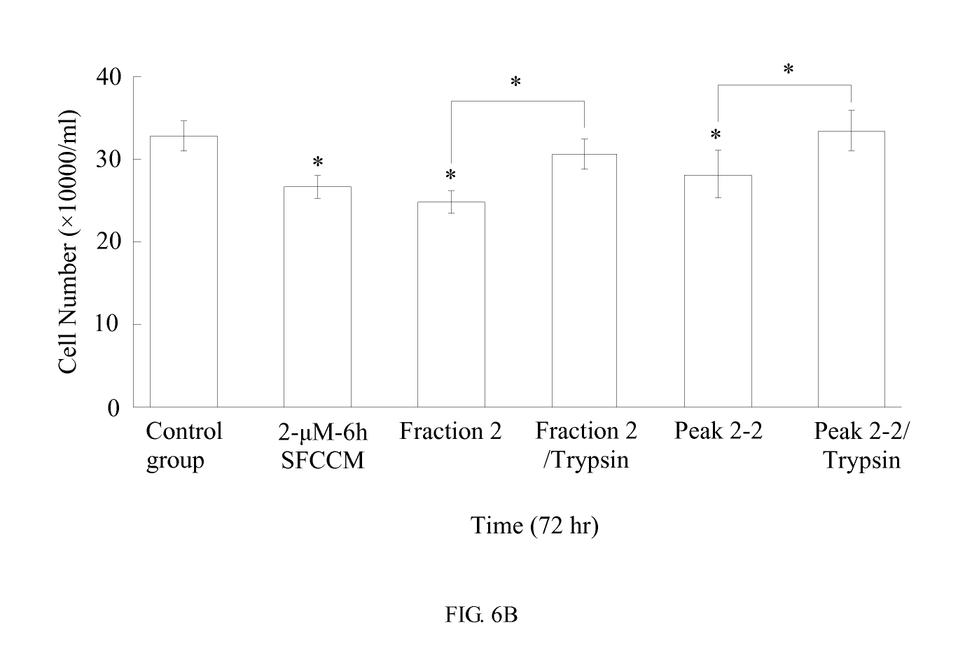

To clarify the biological characteristics of 2-μM-6h-CCM and Peak 2-2 purified substance, the thermal and trypsin digestion processes are carried out. The result shows that both 5% 2-μM-6h-CCM and Peak 2-2 purified substance fail to inhibit proliferation of dermal papilla cells after both processes, as shown in FIGS. 6A and 6B, which show the analytical results of Segment 2 and Peak 2-2 purified substance on thermal stability and trypsin sensibility, respectively. It can be seen that factors secreted from the dermal papilla cells treated with colchicine are protein.

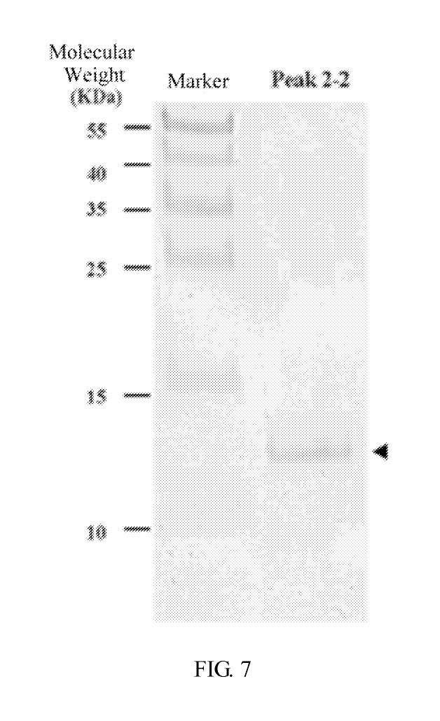

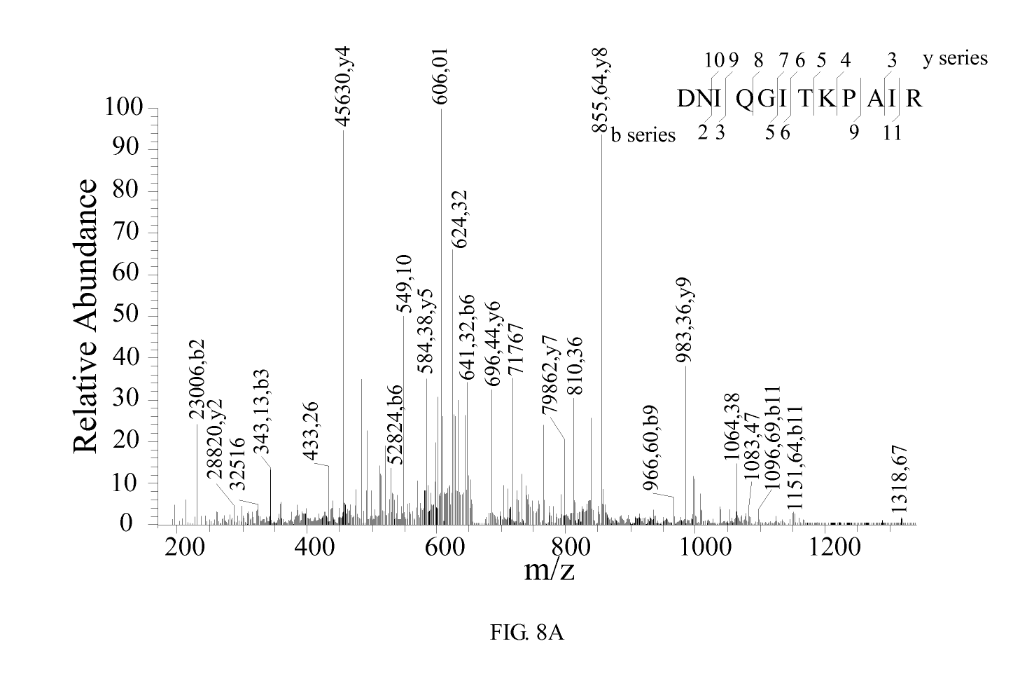

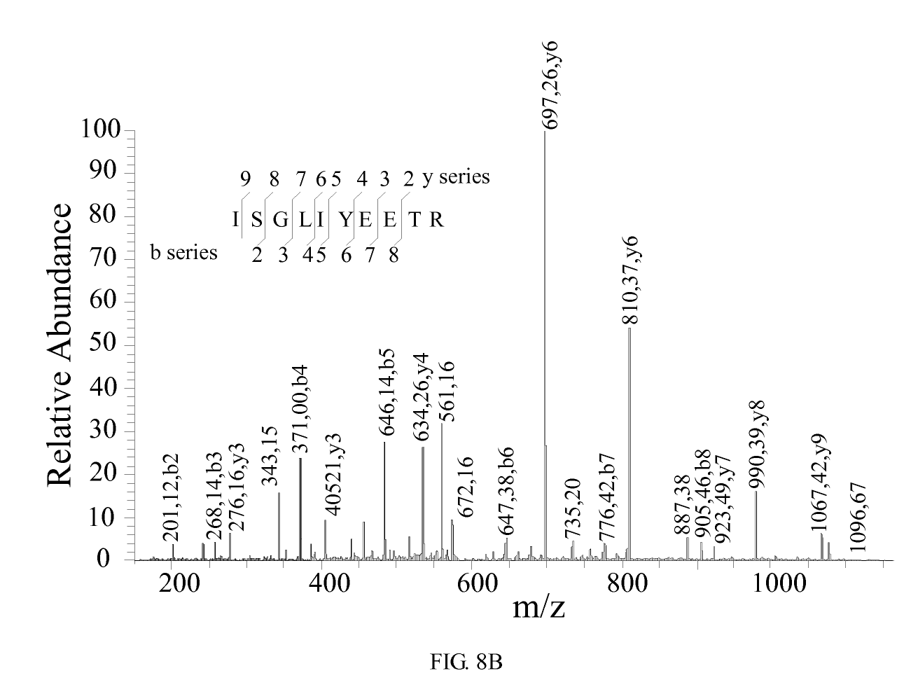

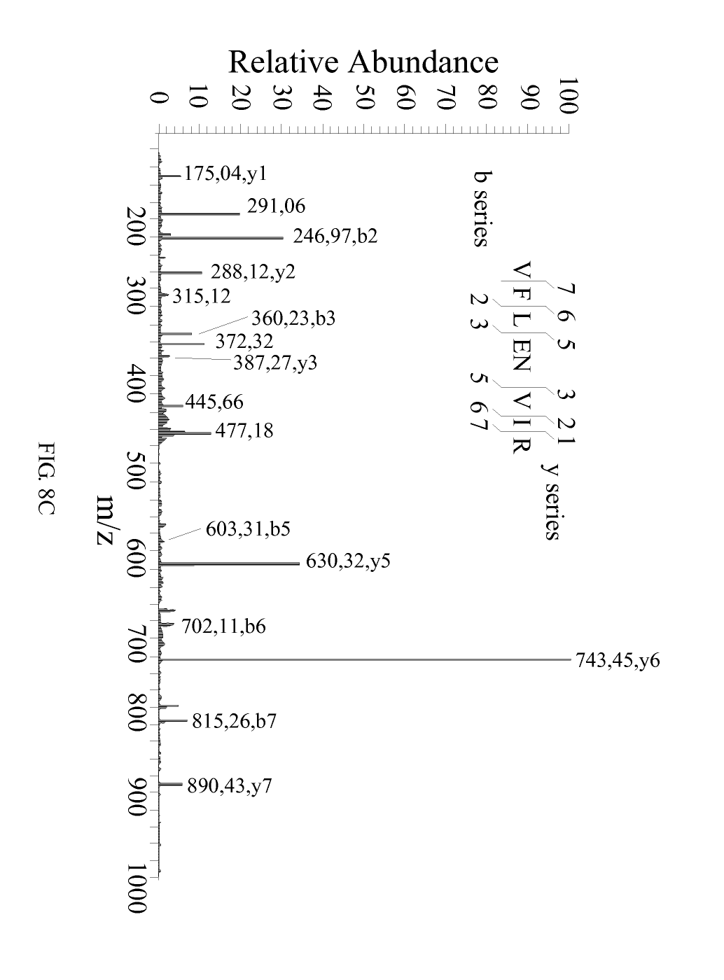

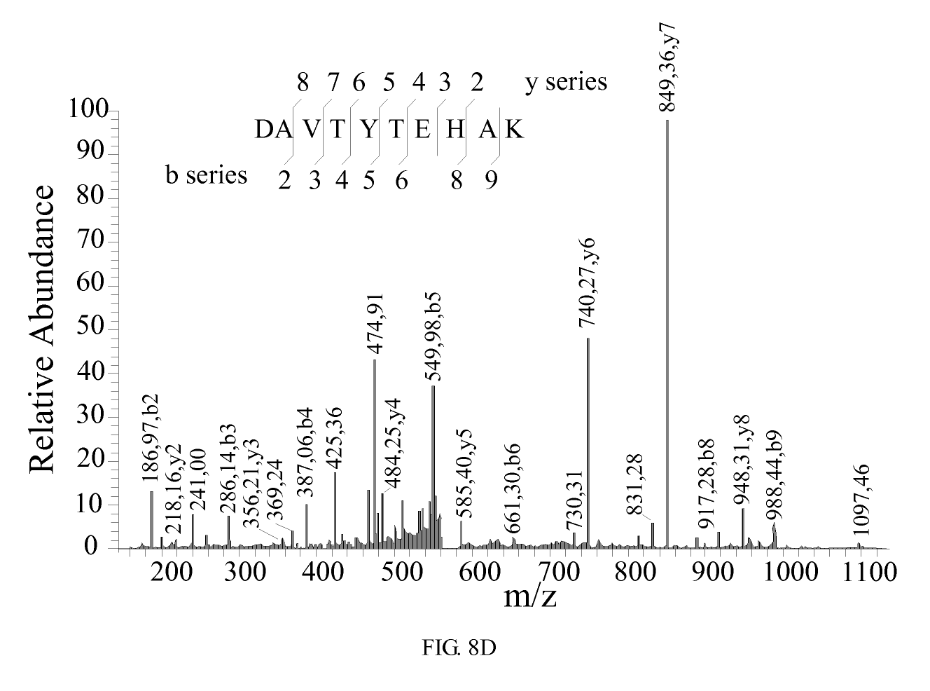

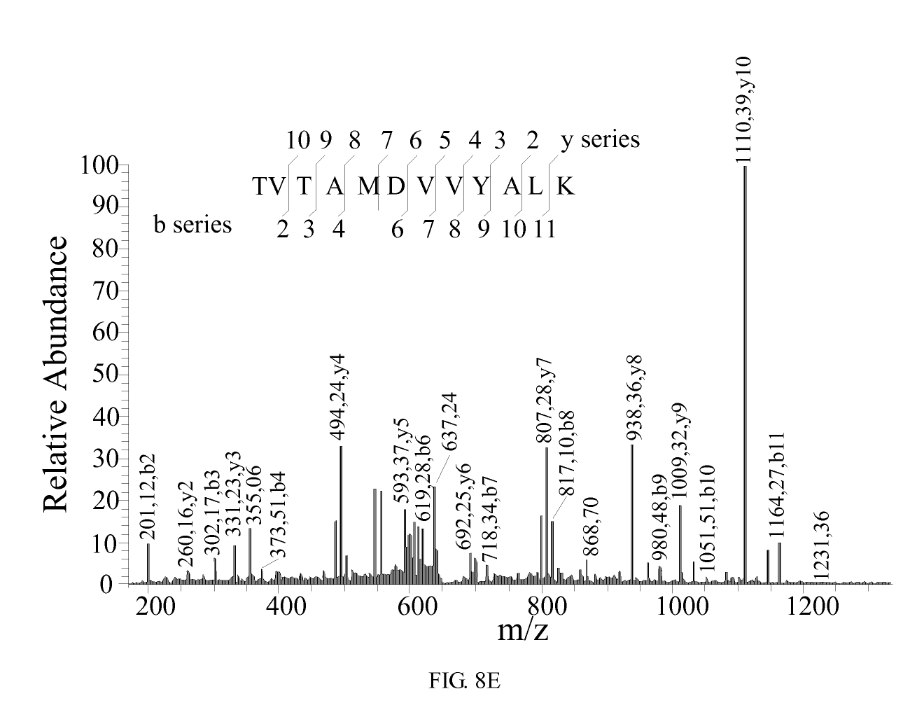

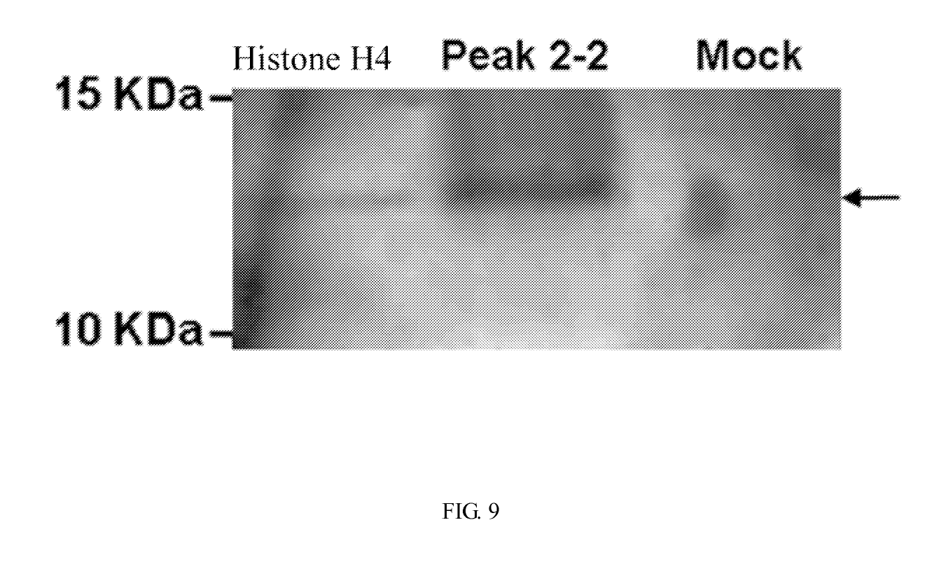

As shown in FIG. 7, Peak 2-2 purified substance is identified as single type protein through 15% SDS-PAGE and silver staining. The protein is recovered, trypsin-cleaved, and analyzed for amino acid sequences with LC-ESI MS/MS to identify Peak 2-2 protein. The result is shown in FIGS. 8A-8E, which are mass spectrum of the trypsin-cleaved fragments. In comparison with Mascot databank, Peak 2-2 protein could be histone H4 (gi|4505821, MW: 11,417 Da, Mowse score: 252, peptide match: 9, sequence coverage: 50%). To identify Peak 2-2 protein as histone H4, Peak 2-2 purified substance is analyzed with polyclonal anti-histone H4 antibody (H-97) by Western blot method, and is confirmed as shown in FIG. 9. From the above, Peak 2-2 purified substance is histone H4.

(8) Protein Function of Histone H4:

Recombinant histone H4 inhibits proliferation of dermal papilla cells directly.

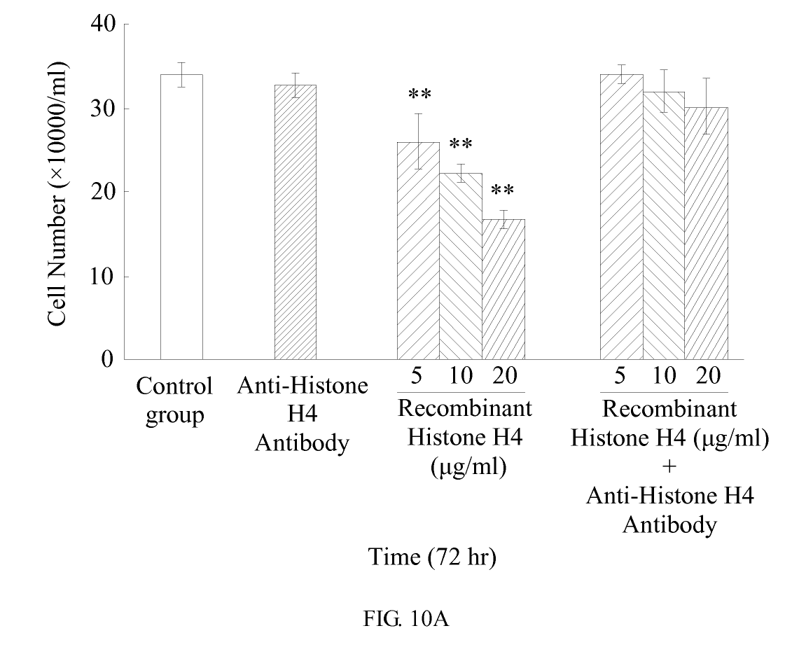

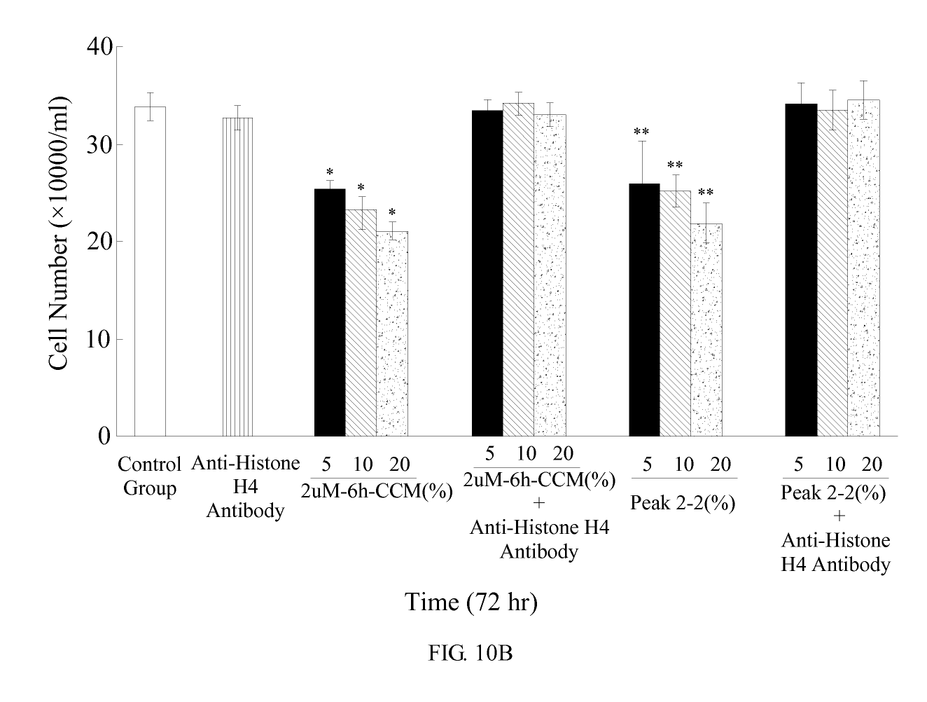

To further confirm that histone H4 protein is capable of inhibiting proliferation of dermal papilla cells, recombinant histone H4 is used. As shown in FIG. 10, the recombinant histone H4 is capable of inhibiting the proliferation, and the inhibition level is proportional to the dosage. However, the inhibition level is reduced by immunoprecipitation with polyclonal anti-histone H4 antibody (H-97). Also referring to FIG. 10B, both 2-μM-6h-CCM and Peak 2-2 purified substance inhibit the proliferation of the dermal papilla cells, and are eliminated by immunoprecipitation with polyclonal anti-histone H4 antibody. It can be seen that the disclosed Peak 2-2 is histone H4 capable of inhibiting the proliferation.

In the above examples, the changes of the protein obtained from dermal papilla cells in conditional medium (secretion plasmid) after stimulated by medicines are analyzed based on proteomics, and the active protein molecules are purified by chromatography and identified as histone H4. Histone H4 not only inhibits proliferation of the dermal papilla cells, but also reduces the alkaline phosphatase activity of the dermal papilla cells.

In summary, because the pharmaceutical composition comprising anti-histone H4 antibody, the effect of histone H4 on the dermal papilla cells can be prevented. That is, the proliferation of the cells is inhibited and the alkaline phosphatase activity is reduced. Thus, the pharmaceutical composition according to the present invention blocks all influence caused by histone H4 on dermal papilla cells and reduces hair loss resulted from medications. Additionally, as the pharmaceutical composition according to the present invention is useful in alleviating the side effects of drug-induced alopecia/effluvium, mental depression caused by hair loss and inadaptability to usual family and social life are moderated, the prognosis effects can be promoted.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A pharmaceutical composition for inhibiting histone H4, comprising an anti-histone H4 antibody and a pharmaceutically acceptable excipient.

2. The pharmaceutical composition for inhibiting histone H4 according to claim 1, wherein the pharmaceutical composition is useful in reducing hair loss resulted from a medication.

3. The pharmaceutical composition for inhibiting histone H4 according to claim 2, wherein said medication comprises colchicine, adriamycin, carboplatin, paciltaxel or taxol.

4. The pharmaceutical composition for inhibiting histone H4 according to claim 1, wherein said pharmaceutically acceptable excipient is selected from a group consisting of flavoring agent, preservative, antioxidant, chelating agent, isotonic agent, tablet adjuvant, colorant, binding agent, and pharmaceutically acceptable carrier.

5. The pharmaceutical composition for inhibiting histone H4 according to claim 1, wherein the pharmaceutical composition is in the form of powder, tablet, emulsion, gel or spray.

6. A manufacturing method of pharmaceutical composition for inhibiting histone H4, comprising a step of mixing an anti-histone H4 antibody and a pharmaceutically acceptable excipient to produce a pharmaceutical composition for inhibiting histone H4.

7. The manufacturing method according to claim 6, wherein said pharmaceutical composition is useful in reducing hair loss resulted from a medication.

8. The manufacturing method according to claim 7, wherein said medication comprises colchicine, adriamycin, carboplatin, paciltaxel or taxol.

9. The manufacturing method according to claim 6, wherein said pharmaceutically acceptable excipient is selected from a group consisting of flavoring agent, preservative, antioxidant, chelating agent, isotonic agent, tablet adjuvant, colorant, binding agent, and pharmaceutically acceptable carrier.

10. The manufacturing method according to claim 6, further comprising a step of making said pharmaceutical composition into the form of powder, tablet, emulsion, gel or spray.
